(19) United States
(12) Patent Application Publication
(10) Pub. No.: US 2015/0148618 A1
(43) Pub. Date: May 28, 2015

(54) Physiological Monitor and Methods for Placement and Wear

(71) Applicants: Krzysztof Sitko, Westhampton Beach, NY (US); George Chen, Hacienda Heights, CA (US); Justin Rubin, Hanover, MD (US)

(72) Inventors: Krzysztof Sitko, Westhampton Beach, NY (US); George Chen, Hacienda Heights, CA (US); Justin Rubin, Hanover, MD (US)

(21) Appl. No.: 14/498,425

(22) Filed: Sep. 26, 2014

Related U.S. Application Data
(60) Provisional application No. 61/883,473, filed on Sep. 27, 2013.
(60) Provisional application No. 61/939,632, filed on Feb. 13, 2014.

(51) Int. Cl. A61B 5/00; A61B 5/11; A61B 5/04; A61B 5/1455; A61B 5/0408; A61B 5/0205

(52) U.S. Cl. CPC A61B 5/6822; A61B 5/04085; A61B 5/02055; A61B 5/04012; A61B 5/0006; A61B 5/0022; A61B 5/14552; A61B 5/6843; A61B 5/7455; A61B 5/7475; A61B 5/1112; A61B 5/002; A61B 5/01
USPC 600/301; 600/382; 600/324

(57) ABSTRACT

Physiological monitors are disclosed, as are systems and methods in which they are used. The physiological monitors are generally horseshoe-shaped and are sized and adapted to fit around the base of the neck. They have forward ends that extend downwardly and inwardly in some embodiments. In systems according to embodiments of the invention, monitors may be wirelessly connected to a device that receives, records, analyzes, and displays physiological and environmental information. Monitors may also be controlled by touch and gestures on touch-sensitive areas of the inner and outer surfaces. In some embodiments, the monitors may be used for long-term, stand-alone monitoring of patients in need of medical monitoring, and allow multiple vital signs, including a three-lead electrocardiogram (EKG) to be recorded from a single location near the base of the neck.

### CROSS-REFERENCE TO RELATED APPLICATIONS

This application claims priority to U.S. Provisional Patent Application No. 61/883,473, filed Sep. 27, 2013, and to U.S. Provisional Patent Application No. 61/939,632, filed Feb. 13, 2014. The contents of both of those applications are incorporated by reference in their entirety.

### BACKGROUND OF THE INVENTION

1. Field of the Invention

In general, the invention relates to physiological and environmental monitors and methods of placement and wear.

2. Description of Related Art

Over the last three centuries, as our understanding of the human body has improved, so has our ability to monitor it. Auscultation—the art of listening to sounds from the body for diagnostic purposes—was refined by Laennaec, who invented the modern stethoscope. The original experiments of Galvani and Volta in “animal electricity” led ultimately to the development of the electrocardiogram (EKG) and the work of Einthoven, who systematized the EKG and described the electrical features of a number of cardiac disorders. More recently, and within the last three decades, pulse oximetry—the measurement of hemoglobin oxygen saturation—has become an indispensible monitoring tool.

Physiological monitoring is now used in a variety of contexts, ranging from the clinical to the prosaic, and techniques that were once confined to research and medical settings for reasons of complexity and cost have found much wider application as their costs have dropped. Whereas Einthoven's EKG involved dipping the limbs into tanks of conductive salt water to read the bioelectrical signals, compact EKG machines with reliable electronics and solid, self-adhesive leads now allow paramedics, and occasionally those with far less medical training, to monitor patients in the field. These techniques have become so much a part of the modern consciousness that many fitness machines, like treadmills and elliptical exercisers, include bioelectrical heart rate measuring sensors so that users can gauge the effects of their exercises.

There have been a number of wearable physiological monitors that include a group of sensors. For example, U.S. Pat. No. 6,836,680 to Kuo, the contents of which are incorporated by reference in their entirety, discloses a detector that picks up EKG, pulse, and vocal sounds. This patent exemplifies many of the difficulties and compromises inherent in creating physiological monitors. Whereas EKG collection usually uses at least 3 electrodes (arranged, as is standard, in the triangular configuration referred to as Einthoven's Triangle), the Kuo device uses only two electrodes, “possibly causing serious interference,” as the reference concedes. The Kuo device is also clamped as a collar around the middle of the neck, a potentially very uncomfortable position for long-term wear. Of course, if a device is uncomfortable to wear or use, the user or patient is less likely to follow a monitoring regimen.

### SUMMARY OF THE INVENTION

One aspect of the invention relates to a physiological and environmental monitor. The monitor has the general shape of an elongated horseshoe and is sized and adapted to be positioned at the base of the neck, seated on or near the clavicles. The monitor may have left and right depending portions that extend slightly downwardly and inwardly. The monitor has at least a main processor, memory, and at least one physiological and/or environmental sensor. In many cases, the monitor will include several sensors of each type. For example, two EKG electrodes may be included in the left and right depending portions of the monitor, with a third electrode located at the rear of the monitor such that it rests against the neck. Environmental sensors may sense quantities like ultraviolet (UV) exposure and the presence and concentration of atmospheric gases and pollutants.

Another aspect of the invention relates to a system for physiological and environmental monitoring. The system comprises a physiological and environmental monitor and a device in communication with the physiological and environmental monitor to collect, analyze, and display data from the monitor. The system may also include one or more server computers communicating with the device via a computer network to provide for long-term storage and comparative analysis of data. In some embodiments, the device may be a smart phone that communicates with the monitor via a communication protocol such as Bluetooth.

Yet another aspect of the invention relates to a method for physiological and environmental monitoring. The method comprises placing a physiological and environmental monitor around the base of the neck of the individual to be monitored. The physiological and environmental monitor has at least one physiological sensor and/or at least one environmental sensor. The method also comprises recording data from the at least one physiological sensor and/or the at least one environmental sensor at regular intervals using an external device.

Another aspect of the invention relates to methods for controlling physiological monitors. In these aspects of the invention, the monitors may include at least one touch-sensitive area on an outer surface thereof, and control of the monitor may be exercised by touch-gestures against the touch-sensitive area. In embodiments according to this aspect of the invention, the inner surface of the monitor may also include a touch-sensitive area, such that proper placement of the monitor on the body is sensed by the monitor and used, for example, to turn the monitor on and off. Additionally, the inner touch-sensitive area may be used as an anti-tamper measure.

A further aspect of the invention relates to an embodiment of a physiological monitor adapted for long-term monitoring of patients. The monitor has the general shape described above, an elongated horseshoe with inwardly extending ends. The monitor has at least an electrocardiography (EKG) circuit and at least one other vital sign sensor. The EKG circuit includes at least three leads and may include a right leg drive (RLD) lead. The sensors may be arranged in two small sensor areas. The sensor areas are opposite one another along the interior perimeter of the monitor, in a position to contact the skin of the sides of the neck. The other vital sign sensors may include a pulse oximeter, a respiration circuit (e.g. an impedance pneumography circuit to measure respiration rate), and an infrared temperature sensor.

In comparison to physiological monitors according to other aspects of the invention, physiological monitors according to this embodiment of the invention typically include a wireless transceiver adapted to connect them with a cellular communication network. Physiological monitors according to this embodiment may or may not include environmental sensors, but may include the touch-based and gesture-based controls, as well as a haptic feedback element, such as a vibrating element, to acknowledge commands. A physiological monitor according to this embodiment of the invention may be used in methods of monitoring patients over relatively long periods of time, such as geriatric patients.

These and other aspects, features, and advantages of the invention will be set forth in the description that follows.

### BRIEF DESCRIPTION OF THE DRAWING FIGURES

The invention will be described with respect to the following drawing figures, in which like features are indicated with like numerals throughout the drawings, and in which:

FIG. 1 is an illustration of a system for physiological and environmental monitoring according to one embodiment of the invention;

FIG. 2 is a front elevational view of a neck-mounted monitoring device according to one embodiment of the invention;

FIG. 3 is a front elevational view of a neck-mounted monitoring device according to another embodiment of the invention;

FIG. 4 is a schematic illustration of the components of a monitor according to an embodiment of the invention;

FIG. 5 is a perspective view of a monitor according to another embodiment of the invention;

FIG. 6 is a perspective view of a monitor according to yet another embodiment of the invention;

FIG. 7 is a schematic illustration of the sensor areas of the monitor of FIG. 6; and

FIG. 8 is a schematic illustration of the components of the monitor of FIG. 6.

### DETAILED DESCRIPTION

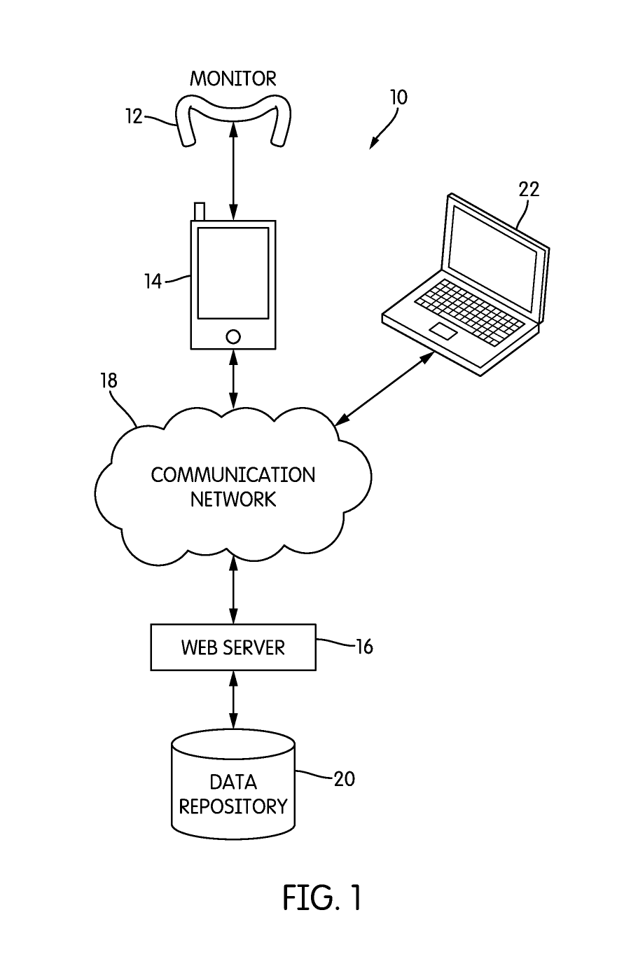

FIG. 1 is an illustration of a system, generally indicated at 10, for physiological and environmental monitoring. The system comprises a wearable monitor 12 that is designed to be worn by an individual to be monitored and an external data logging and display device 14 that is adapted to communicate with the monitor 12, typically using a local wireless communication protocol.

Generally speaking, the wearable monitor 12 is adapted to be worn by an individual around the base of the neck, seated just above the clavicles, as will be described below in more detail. The wearable monitor 12 typically includes at least one physiological sensor that is adapted to sense a vital sign or physiological state of the individual, and at least one environmental sensor adapted to sense a characteristic or characteristics of the environment around the individual. The vital sign measured by the physiological sensor and the environmental characteristic measured by the environmental sensor may or may not have a known scientific correlation with one another, depending on the embodiment.

Examples of physiological sensors in the monitor 12 include temperature sensors, electrocardiogram (EKG) electrodes, pulse oximetry sensors, electromyelogram (EMG) electrodes, electroencephalogram (EEG) electrodes, galvanic skin response (i.e., skin conductivity) sensors, pneumography sensors, and microphones for auscultation. Ultimately, any type of physiological sensor that can produce an accurate reading from the position in which the monitor 12 is worn may be included.

Examples of environmental sensors include ultraviolet (UV) light detectors, general photodetectors, environmental noise sensors or microphones, humidity sensors, gas and vapor sensors, ambient temperature sensors, and altimeters. Gas sensors may include common atmospheric gases and pollutants (e.g., oxygen, ozone, carbon monoxide, carbon dioxide, nitrogen, sulfur dioxide, nitrogen oxides, and volatile organics (VOCs)), chemical contaminants (e.g., hydrogen fluoride, hydrogen chloride, sodium hydroxide), and poisons or toxins (e.g., phosgene, sarin gas, cyanides, arsenic, etc.).

In most embodiments, the monitor 12 will include several physiological sensors and several environmental sensors, and may include any number of either, limited only by the form factor of the monitor 12 and the general desirability of limiting the weight of the monitor 12 so that it can be worn comfortably over long periods of time. The monitor 12 will also generally include sufficient onboard processing capabilities to gather data from whatever sensors are present. The monitor 12 may also have an onboard cache or storage memory, e.g., 1-2 GB of flash memory. However, in the illustrated embodiment, most data logging and analysis is done by the external device 14 or by other computer systems in communication with it.

In some embodiments, the device 14 may be a dedicated device with hardware and software adapted to log and analyze data from the monitor 12, either continuously, at regular intervals, or as necessary. In other embodiments, the device 14 may be a multipurpose device, like a smart phone, tablet computer, laptop, or other general-purpose computing device with software (such as an application or “app”) that allows the device 14 to communicate with the monitor 12 to log and analyze data from the monitor 12.

The connection between the monitor 12 and the device 14 may be a wired, physical connection via standard input/output ports (e.g., a USB port on the monitor 12 and the standard dock/connector interface on the device 14). However, in particularly advantageous embodiments, the connection between the monitor 12 and the device 14 will be a wireless connection. The type of wireless connection will vary from embodiment to embodiment. If the device 14 is a multipurpose device, like a smart phone or tablet, the wireless communication protocol will generally be one available on the device 14. For example, Bluetooth, IEEE 802.11a/b/g/n (WiFi), WiFi Direct, and cellular data communication protocols may all be used, with Bluetooth being a particularly advantageous communication protocol in at least some embodiments. Other protocols, like near-field communication (NFC) may be used to pair a particular smart phone or tablet computer with the monitor 12 to act as the data logging device 14 and/or to initiate other, higher-bandwidth communication protocols.

On the other hand, if the device 14 is a dedicated device specifically intended to communicate with, log data from, and manage the monitor 12, the wireless protocols used may be virtually any known wireless protocols, including those not typically found in a smart phone, like IEEE 802.15 (ZigBee). In some embodiments with a dedicated, special-purpose device 14, the frequency bands used for communication may be those reserved for use by medical devices. Of course, the actual communication protocols that are used in any embodiment will depend on a number of factors, including the frequency with which data is collected, the amount of on-board buffer or data storage on the monitor 12, the bandwidth necessary to communicate data from the monitor 12, the communication range, and the power consumed by the communication protocols.

As is also shown in FIG. 1, the device 14 may be in communication with one or more servers 16, such as World Wide Web servers, through a communication network 18, such as the Internet. The server or servers 16 are in communication with one or more data repositories 20 for long-term data storage and more complex analysis. In other words, system 10 may be a “distributed” and “cloud-based” data gathering and processing system in at least some embodiments. The monitor 12 gathers data, provides for short-term storage of data, and usually performs preliminary processing tasks, which may include signal filtration as well as compression tasks, like feature extraction. The data is then downloaded to the device 14, which may provide more sophisticated processing, if necessary, and also provides user display, analysis, and interface functions. Communication between the device 14 and the server or servers 16 allows for longer-term storage, analysis, and, in some cases, comparisons with other individuals who are also being monitored.

As is also shown in FIG. 1, other computing devices, like a laptop computer 22, may communicate with the web server 16 and gain access to the data from the monitor 12. If system 10 does include “cloud-based” or remote server features, as is the case in FIG. 1, authentication and encryption protocols may be used to ensure that only individuals authorized to view monitoring data are able to do so. For example, each individual being monitored by a monitor 12 could sign up for an account with a service that provides secure access to the monitoring data through the server 16. If the monitoring was prescribed or is being used by medical professionals in diagnosis or treatment, they could also be provided with accounts for accessing data from their own patients.

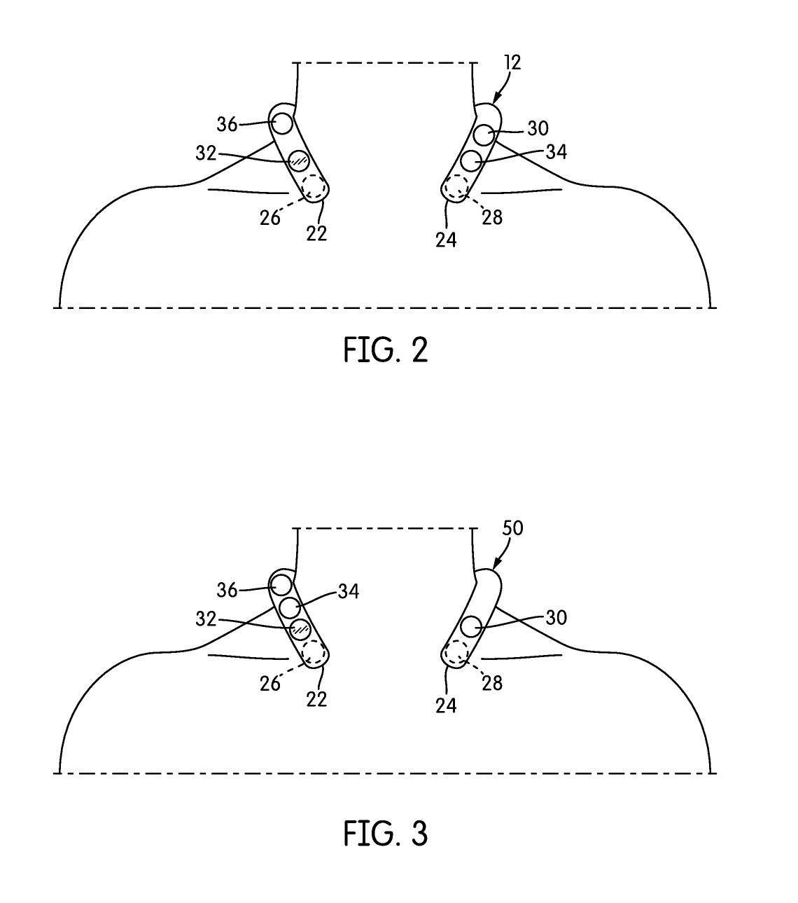

FIG. 2 is a front elevational view of a monitor 12 as worn on a patient. The monitor 12 has the general shape of an elongated horseshoe and fits around the base of the neck. As shown, the front ends 22, 24 of the monitor 12 angle downwardly and inwardly (i.e., medially, with respect to the wearer), terminating about at or just below the level of the clavicles. The monitor 12 would typically include either an internal resilient member that allows it to clamp around the neck, a telescoping mechanism that allows its circumference to be changed, or other kinds of mechanisms that allow it to adjust to different size necks and to remain in place on those necks for moderately long periods of time (e.g., from one to several hours). Overall, the position in which the monitor 12 is worn is intended to be as comfortable as possible.

In a typical monitoring situation, the position of some sensors on the body is critical to their functioning, while the positioning of other sensors is not. Of the position-critical sensors, the classic example is EKG electrodes. Failure to place EKG electrodes correctly may result in either a total failure to read an electrocardiographic signal or the reading of a signal along a different electrical axis than what was intended, leading to confusing data. EMG and EEG electrodes are generally also position-critical. Sensors that are not position-critical include sensors reading vital signs like body temperature, which can be taken essentially anywhere on the body. With sensors whose position is not critical, a calibration process or conversion process can often be used to normalize the data for comparisons with typical medical data.

Most environmental sensors are not position critical, so long as a basic rule or rules are observed. For example, light and UV sensors should be positioned along the exterior of the monitor 12, where light will strike them.

Despite the importance of positioning certain sensors correctly and well, it may be advantageous to compromise sensor position somewhat in order to achieve more comfort in the wear of the monitor 12, and thus, more ability to monitor the individual over the long term. For example, as was described briefly above, the Kuo patent discloses mounting a monitor essentially at the vertical center of the neck, which may be an optimal location for EKG electrodes. However, that position is not necessarily very comfortable. By contrast, the present inventors have found that acceptable EKG readings can be taken from the base of the neck with far more comfort.

In the illustration of FIG. 2, one EKG electrode 26, 28 is provided in each of the front ends 22, 24 of the monitor 12 with the electrodes facing inwardly and in contact with the skin. The two front electrodes 26, 28 serve as the standard right arm (RA) and left arm (LA) electrodes in Einthoven's Triangle. A third electrode, not shown in FIG. 2, is provided in the rear of the monitor 12 such that in the view of FIG. 2, it would be centered on the back of the neck. This third electrode acts as the left leg (LL) electrode, which serves as the common “ground” electrode for the other two. Depending on which set of two electrodes are being used for measurement at any one time, this arrangement provides standard EKG Leads I, II, and III. FIG. 2 also schematically illustrates the locations of an SpO2 (pulse oximetry) sensor 30, a UV sensor 32, a gas sensor 34, and a body temperature sensor 36. These represent an exemplary suite of sensors that might be included in a monitor 12.

Those sensors that are not position critical may be arranged in any convenient way within the monitor. In the monitor 12, the UV sensor 32, gas sensor 34 and temperature sensor 36 are all on the right side of the patient's neck, with only the pulse oximetry sensor 30 and one of the EKG electrodes 28 on the left. This is only one possible arrangement. As another example, FIG. 3 illustrates a monitor 50 in which the EKG electrodes 26, 28 are in the same place, with the temperature sensor 36 and UV sensor 32 on the right side of the patient's neck and the gas 34 and pulse oximetry sensor 30 on the left.

The internal components of a monitor 12, 50 according to embodiments of the invention may vary considerably depending on the type and number of sensors that are installed. In some embodiments, a single processor, such as a microcontroller unit, may manage all of the functions of the monitor 12, 50. In other embodiments, a master processor may manage the overall function of the monitor 12, 50 and communicate with a number of dedicated processors that take data from the individual sensors. These dedicated processors may be, for example, processors that require less power than the main processor, so that they can take data more frequently without consuming as much power.

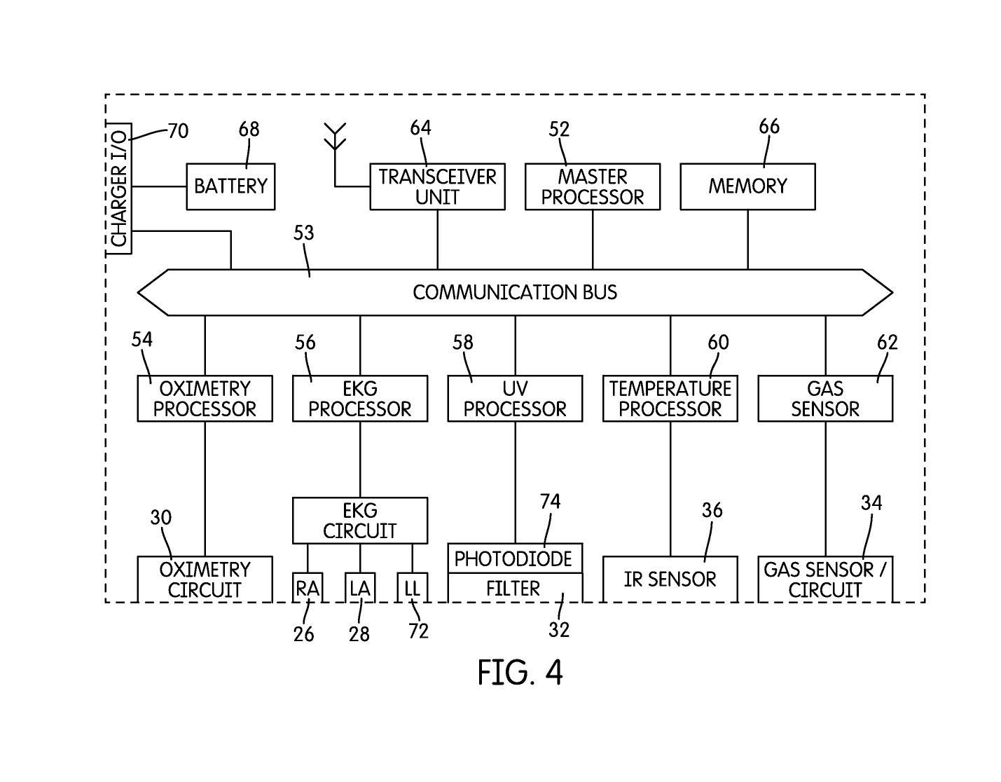

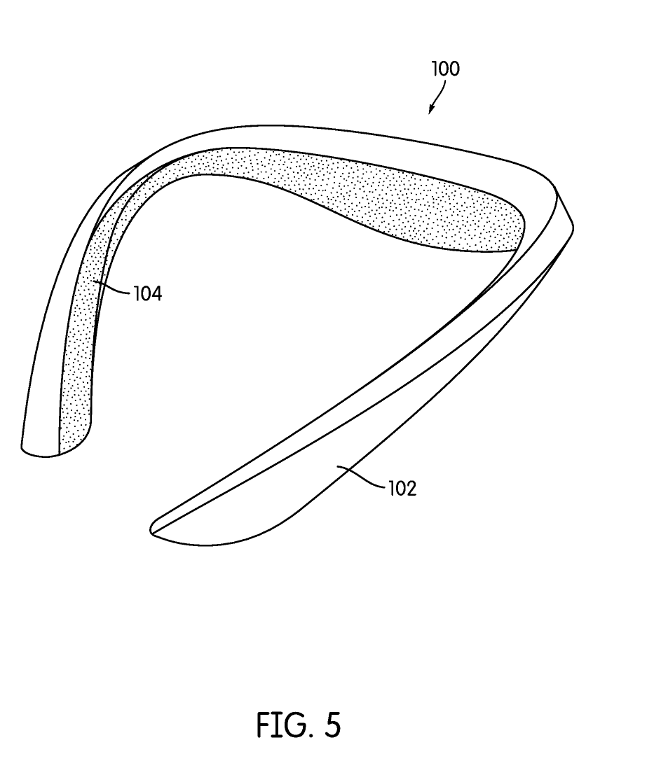

FIG. 4 is a schematic illustration of one configuration of the internal components of a monitor 12, 50. As described above, the illustrated configuration includes a master processor or controller unit 52 and a number of dedicated, task-specific processors 54, 56, 58, 60, 62 in communication with the master processor 52 that handle data collection for the individual sensors. In one embodiment, the master processor may be, for example, an MSP430 16-bit microcontroller (Texas Instruments, Inc., Dallas, Tex.). The dedicated processors 54, 56, 58, 60, 62 are typically processors that are more application-specific and require less power, such that the monitor 12 can collect more data using less power.

As shown in FIG. 4, the master processor 52 communicates with the dedicated processors 54, 56, 58, 60, 62 by means of a communication bus 53. Within the master processor 52, an interface such as a serial peripheral interface is used to receive data and to communicate.

The master processor 52 is coupled to a transceiver unit 64, such as a Bluetooth transceiver unit, through the bus 53, although in some embodiments, a transceiver unit 64 may be housed with or integrated into the processor 52. Multiple transceiver units may be included in the monitor 12, 50 if it is to be compatible with multiple communication protocols.

The monitor 12, 50 also includes memory 66. Although the memory 66 is shown as a singular element, several different types of memory may be included in monitors 12, 50 according to embodiments of the invention. The master processor 52 and the other processors 54, 56, 58, 60, 62 may have their own onboard cache memories, and may also communicate with the memory 66. Typically, the memory 66 installed in a monitor 12, 50 would include random access memory (RAM) and Flash memory or a solid state drive (SSD) for intermediate-term data storage.

A battery 68 is also included as a power source. The battery may be, for example, a lithium ion battery. The battery 68 is connected to a charging circuit 70, which may also provide input/output (I/O) functions in some embodiments, and if it does, may communicate with the master processor 52 through the bus 53 for that reason. For example, the charging circuit 70 may provide an external electrical connector for charging. In embodiments where the charging circuit also provides for I/O, the circuit 70 may include a connector such as a Universal Serial Bus (USB) or mini-USB port for both charging and I/O functions. Of course, a custom type of connector that allows for both charging and I/O may be used.

In the illustration of FIG. 4, the bus 53 provides for communication among the various elements of the monitor 12, 50. However, in other embodiments, some or all of the elements could be directly connected to the master processor 52 itself.

Each of the sub-processors 54, 56, 58, 60, 62 communicates with the master processor 52 via the bus 53 and is powered by the battery 68. As those of skill in the art will appreciate, each type of sensor present in the monitor 12, 50 may include its own data acquisition circuit, the details of which are not shown in FIG. 4. Each processing circuit may include, e.g., amplifiers, filters, and an analog-to-digital converter that enables the corresponding processor 54, 56, 58, 60, 62 to read the data. Generally speaking, data acquisition circuits for most common physiological sensors are well known in the art, and any may be used in embodiments of the invention. Specific considerations for individual sensors will be described in more detail below.

The oximetry processor 54 and associated circuit may use, for example, a VBPW34S photodiode (Vishay Semiconductor Opto Division, Shelton, Conn.), 650 nm red and 940 nm infrared LEDs, and an AFE4400 processor/front end (Texas Instruments, Inc., Dallas, Tex.).

The EKG processor 56 may be an ADS1292 analog front end for EKG (Texas Instruments, Inc., Dallas, Tex.). The three EKG electrodes, including the right and left electrodes 26, 28, positioned at the front of the monitor 12, 50, and the left leg electrode 72, positioned in the center rear of the monitor 12, 50, against the back of the neck may be, for example, Plessey PS25454 electrodes (Plessey Semiconductors, Ltd., Plymouth, United Kingdom).

Generally speaking, a UV detector would include a photodiode 74 or other photosensor sensitive in the UVA and UVB frequency ranges and an optical filter 76 that filters the incoming light such that only those frequencies pass to the photodiode 74.

The temperature processor 60 and sensor 78 may comprise an infrared detector that finds the difference between the ambient temperature and the temperature of the skin that it faces. One example is an MLX90614 infrared thermometer (Melexis Technologies NV, leper, Belgium).

The processor 62 for the gas sensor or sensors, and the nature of the circuit that is connected to it, will depend on the nature of the gases that are being detected. One suitable example is a MICS-4514 metal oxide semiconductor gas sensor (SGX SensorTech Ltd., Essex, United Kingdom) that is adapted to detect carbon monoxide, nitrogen dioxide, hydrocarbons, ammonia, and methane.

While FIG. 4 and parts of the description above may assume that each vital sign, physiological characteristic, or environmental characteristic is measured by a sensor and reported quantitatively, in some embodiments, some characteristics may be inferred or derived from the data from other sensors. For example, EKG can be used to establish heart rate, which may be reported separately. Moreover, the quantitative data from some sensors may be used to make qualitative or more general determinations. For example, a UV sensor may be used to determine whether an individual being monitored is indoors or outdoors based on the level of UV exposure, as compared with defined thresholds for indoor and outdoor environments. Additionally, general-purpose sensors, like 3-axis accelerometers, may be installed to provide a variety of positional information, and may also allow the monitor 12 to act as a pedometer. Where a general or qualitative determination is being made based on sensor data, that determination may be made either by the monitor 12, 50 or by software routines running on the device 14.

Although not shown in FIG. 4, the monitor 12, 50 may also include a display element, such as a light-emitting diode or diodes (LEDs) or a full display to communicate status information to the user. Additionally or alternatively, it could include a speaker that provides audio prompts. However, in some embodiments, the monitor 12, 50 may simply communicate status information to its paired device 14, and the device may then handle communicating that status information to the user.

In other embodiments, controls may be built directly into the monitor. FIG. 5 is a perspective view of a monitor, generally indicated at 100, according to another embodiment of the invention. While a monitor 12, 50, 100 may include any number of standard buttons, switches, sliders, or other conventional controls, monitor 100 is equipped with one or more capacitative sensors, such that areas of the surface or surfaces of the monitor 100 are responsive to touch or gesture. This allows a user to control the monitor 100 and may, in some cases, entirely replace a device 14 as a means of controlling the monitor 100. Moreover, while capacitative touch sensing is one means of detecting touch, any means of sensing touch may be used.

The monitor 100 has the same general shape as the monitors 12, 50 of other embodiments. The monitor 100 also has both an outer touch-sensitive area 102 and an inner touch-sensitive area 104. The two touch-sensitive areas 102, 104 may cover the entire outer and inner surfaces of the monitor 100 or only portions of those surfaces. In some cases, a monitor 100 may have only an outer touch-sensitive area 102 or an inner touch-sensitive area 104. The monitor 100 may also include entertainment features, like the ability to store and play music, or the ability to act as a BLUETOOTH® receiver/headset for a device 14 that stores and plays music. In some embodiments, the monitor 100 may include a standard headphone jack.

As one example of how the outer touch-sensitive area 102 may be used to control the monitor 100, tapping on one side of the monitor 100 may increase the volume by 5%, while tapping on the other side of the monitor 100 may decrease volume by 5%. Double-tapping either side of the outer surface of the monitor 100 may pause or play music. Swiping forward on one side of the monitor 100 may cause the monitor 100 to skip to the next song if music is being played, while swiping forward on the other side of the monitor 100 may cause the monitor to skip to the beginning of the current song or back to the previous one. Meanwhile, if a user rests one or two fingers against the left or right side of the monitor 100, similar to how one might check his or her pulse, the monitor 100 may play an auditory message indicating the current readings of any installed sensors, or data derived from the sensors.

While the monitor 100 is in use, the inner touch-sensitive area 104 will generally be inaccessible to the fingers. However, the inner touch-sensitive area 104 may be used to turn the monitor 100 on and off, such that the monitor 100 turns on when the inner touch-sensitive area 104 registers skin contact and turns off when, or shortly after, the inner touch-sensitive area 104 no longer registers skin contact. Of course, the monitor 100 may be programmed to turn on or off only when a certain percentage of the inner touch-sensitive area 104 registers contact, in order to ensure that the contact is with the neck and not with, for example, the fingers.

The inner touch-sensitive area 104 may also be used to ensure that the monitor 100 is properly placed for data acquisition and as an anti-tampering measure. For example, if the monitor 100 is equipped with an accelerometer and adapted for use as a pedometer, a user seeking to register more steps might try shaking the device rhythmically in an attempt to trigger the accelerometer to register that the user is walking However, if the inner touch-sensitive area 104 must register skin contact for the monitor 100 to be on and the sensors to be reading data, then this kind of tampering becomes much more difficult.

The monitor 100 may also include the same sorts of environmental sensors described above, but in at least some embodiments, it may not include environmental sensors.

### Methods of Use

Monitors 12, 50 according to embodiments of the invention may be used in any number of ways and for any number of purposes. Typically, any embodiment will begin when a person to be monitored puts on a monitor 12, 50 and seats it at the base of the neck, as illustrated in FIGS. 2 and 3. Indicia may be provided on the monitor 12, 50 that illustrate its proper position on the body graphically.

Either before or after it is placed, the monitor 12, 50 is paired or placed in communication with a device 14. Once seated and activated, the monitor 12, 50 may begin any necessary initialization and/or calibration steps. In some embodiments, if the monitor 12, 50 captures data that is outside of pre-set limits for more than a predefined amount of time, the monitor 12, 50 or its device 14 may alert the user to reposition the monitor 12, 50.

The frequency with which data is taken and reported once the monitor 12, 50 is in operation will depend on a number of factors, including the nature of the sensors, the amount of power available, and the context in which the monitor 12, 50 is being used. In a typical embodiment, the monitor 12, 50 might take a complete set of readings about once a minute or once every few minutes. If the device 14 is on and within range, the data might be transmitted directly to the device 14 with only temporary storage in the onboard memory 66 of the device 12, 50.

In a typical embodiment, the device 14 may perform additional smoothing, averaging, filtering, or other processing steps on incoming data from the monitor 12, 50 before it is displayed or used. In medical monitoring contexts, it may be important to store all of the data that is gathered for later or more complex analysis. In that case, the device 14 may store or back up data in the data repository 20 by connecting with the web server 16 via the communication network 18. In more general contexts, such as when using a monitor 12, 50 to monitor athletic performance, the device 14 may average the data and present the average readings for a particular period of time, such as the average reading for the last half hour or hour, the average reading for the last week, the average reading for the last year, etc. In some cases, the full data set may be uploaded to the data repository 20, while in other embodiments, excess data may simply be deleted.

### Long-Term Monitoring

As is evident from the above description, one particular advantage of monitors 12, 50, 100 according to embodiments of the invention is that a variety of vital signs and, if desired, environmental data can be gathered from a single location on the body using a device that is relatively comfortable to wear—without the need for sensors positioned elsewhere or wires that extend over the body. For those reasons, monitors 12, 50, 100 according to embodiments of the invention may be particularly suitable for long-term monitoring of patients. In some embodiments, this monitoring may take place in hospitals, clinics, and long-term care environments.

Monitors 12, 50, 100 according to embodiments of the invention may also be particularly useful in monitoring patients outside of hospitals and other care facilities. For example, a generally stable geriatric patient may wear a monitor 12, 50, 100 on an ongoing basis. Data generated by the monitor 12, 50, 100 may be transmitted through a device 14 to a server 16 and data repository 20 as illustrated in FIG. 1.

However, in many embodiments, it may be more convenient if the monitor 12, 50, 100 is configured to operate alone—without a local device 14 wirelessly connected. In these embodiments, the transceiver unit 64 built into the monitor 12, 50 may be configured to communicate with cellular communication networks using, for example, GSM/EDGE, UTMS/HSPA+, DC-HSDPA, or CDMA EV-DO protocols, depending on the location of the monitor 12, 50, 100 and the cellular networks operating in the area. In some cases, monitors 12, 50, 100 may alternatively be configured to use frequency bands and communication protocols set aside for medical or first-response communication.

When a cellular network is the communication network 18, the monitor 12, 50, 100 may send data either continuously or at intervals. Generally, the frequency with which data is sent will seek to balance the need for adequate monitoring with the amount of battery power available and the amount of memory available on the monitor 12, 50, 100 for temporary storage.

In some cases, the monitor 12, 50, 100 may transmit data at regular intervals and also when a material change in condition is detected, such as a decline in heart rate below a defined threshold or an arrhythmia. Triggers for sending data could also be based on oxygen saturation or on any other vital sign measured or derived by the monitor 12, 50, 100—for example, an alarm could be established and data transmitted if the patient's oxygen saturation falls below 90%. Of course, many different algorithms may be used in various embodiments of the invention.

Although any embodiment of monitor 12, 50, 100 could be used in a long-term monitoring situation, a monitor similar to monitor 100 of FIG. 5 may be particularly useful, because its inner touch-sensitive area 104 can be used to determine whether the monitor 100 is properly placed and can trigger an alarm if the monitor 100 falls or is moved out of the correct position.

In the monitors 12, 50, 100, described above, the physiological sensors and environmental sensors, if present, may be in the same basic locations illustrated in FIGS. 2 and 3. However, the present inventors have found that the broad (side) portions of the neck are also suitable for the placement of physiological sensors that require skin contact, like EKG electrodes, physiological temperature sensors, and pulse oximetry sensors, because monitors 12, 50, 100 may have more reliable skin contact in that area. Moreover, it has been found that grouping several sensors within a relatively small area, rather than spacing them around the inner perimeter of the monitor 100, may be advantageous.

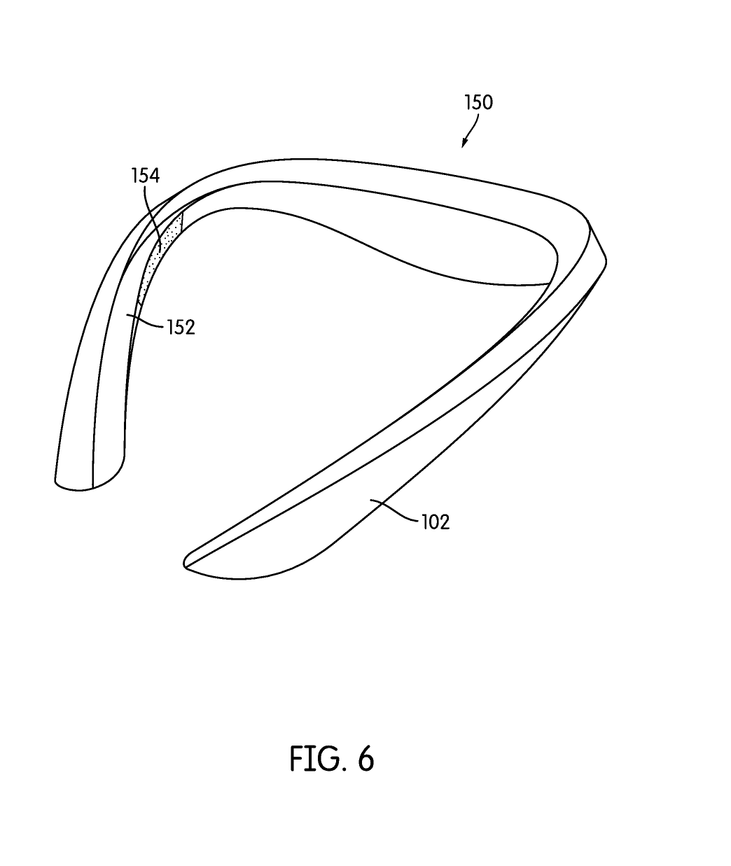

FIG. 6 is a perspective view of a monitor 150 that includes many of the features of the monitors 12, 50, 100 described above and that is particularly suitable for long-term monitoring. On the inner, skin-facing surface 152 of the monitor 150, a sensor area 154 lies along the inner perimeter of the monitor 100, positioned to contact the side of the neck. An additional sensor area 156 (not shown in FIG. 6) lies directly opposite the sensor area 154, positioned to contact the other side of the neck.

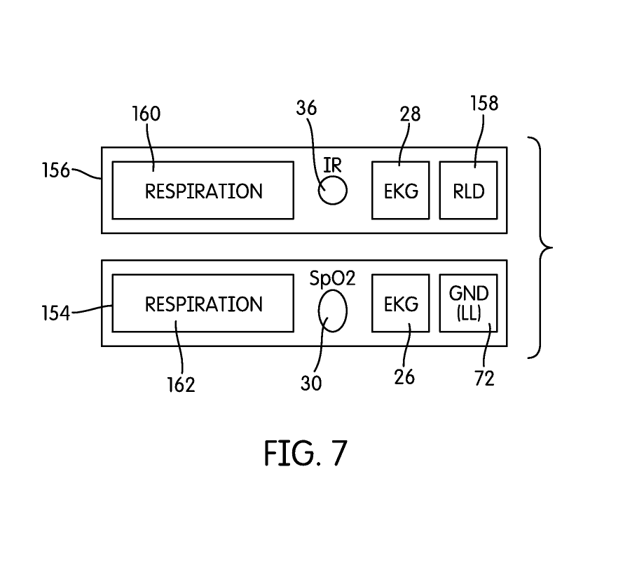

As was described above in great detail, the sensor complement on any particular embodiment of monitor 12, 50, 100, 150 may vary, depending on its intended use and other factors. The monitor 150 has a sensor complement particularly adapted for long-term physiological monitoring, although in other cases, it may incorporate any of the sensors described above, or any other physiological or environmental sensors necessary to accomplish its purpose. FIG. 7 provides schematic views of the layouts of the sensor areas 154, 156.

Each sensor area includes two EKG-related electrodes. The sensor area 154 includes the RA electrode 26 and the LL or ground electrode 72. The sensor area 156 includes the LA electrode 28 and a right leg drive (RLD) electrode 158. One of the difficulties with electrocardiogram measurement is noise, particularly common-mode noise, which affects all of the electrodes 26, 28, 72. The usual source of common-mode noise is the electrical power grid—for example, noise with a frequency of 60 Hz is common in the United States, where the AC power grid runs at a frequency of 60 Hz. In the monitor 150, the EKG circuit 161 includes a common-mode noise reduction system that detects common-mode noise, inverts the signal, and uses the RLD electrode 158 to inject that inverted signal into the body in order to cancel it out. (This does result in very small amounts of current being injected into the body.)

In addition to the EKG-related electrodes 26, 28, 72, 158, the monitor 150 is equipped with an impedance pneumography circuit that allows the monitor 150 to detect respiration rate based on changes in electrical impedance as the wearer breathes. Two respiration electrodes 160, 162 are provided for this purpose, one in each sensor area 154, 156. One sensor area 156 also includes an IR sensor 36 to measure patient temperature, while the other sensor area 154 includes a pulse oximetry sensor 30.

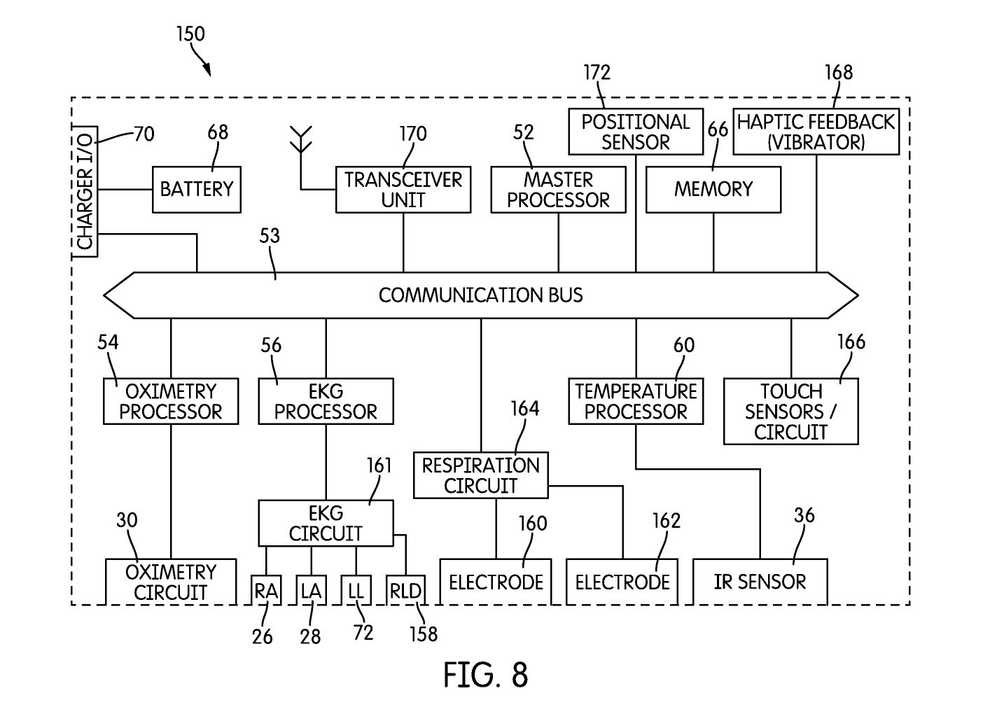

FIG. 8 is a schematic illustration of the monitor 150. The monitor 150 includes many of the components of the monitors 12, 50, 100 described above, including a master processor 52, a communication bus 53, a memory 66, a battery 68 and a charging and/or input-output port 70. However, as was described above, the configuration of its sensors differs somewhat from the other embodiments.

Like the other embodiments, the monitors 12, 50 has at least a three-lead EKG, including the standard electrodes 26, 28, 72 and an EKG processor 56. In this case, the RLD electrode 158 is also shown in FIG. 8. An oximetry circuit 30 and oximetry processor 54 are also included, and an infrared sensor 36 and temperature processor 60 may be included as well. In the monitor 150, if present, the IR sensor 36 may be positioned to read the patient's own skin temperature. In addition to those components, a respiration circuit 164 is provided to measure respiration rate by impedance pneumography, as was described above, and is coupled to the two electrodes 160, 162 used for that purpose.

The monitor 150 also includes one or more touch sensor circuits 166 to read and control the kinds of touch-sensitive areas 102, 152 that were described above. While some embodiments of a monitor like monitor 150 may include entertainment features, and the touch-sensitive areas 102, 152 may be used to control those features, in most embodiments, the touch-sensitive areas 102, 152 will exclusively or also be used to control the medical and monitoring features. For example, using a specific gesture or touching a specific area may cause the monitor 150 to immediately report its data, and using another type of gesture or series of gestures may initiate an alarm that requests medical assistance. The identification and processing of touch gestures is well known in the art, and any appropriate gestures may be used.

The monitor 150 may include any of the interface and input-output elements described above, including a small screen, LED indicators, a speaker, and other conventional elements or devices. As shown in FIG. 6, the monitor 150 includes a haptic feedback element 168 which, in this case, is a vibrating element. The vibrating element 168 may be triggered in response to changes in state and user commands in order to confirm to the user that the commands and changes in state have been accepted. For example, the monitor 150 may be caused to vibrate after it is turned on, after the user manually instructs it to report data using touch, a touch gesture, or series of gestures, and when the measured or calculated vital signs cause an alarm.

As was described above, the transceiver unit 170 may also be different than the transceiver unit 64 of other embodiments, insofar as the transceiver unit 170 is adapted to connect the monitor 150 to cellular networks.

In addition to the above components, as was described briefly above with respect to the monitor 100, the monitor 150 may include one or more positional sensors 172 whose purpose is to ascertain the position and/or orientation of the monitor 150 in space. Positional sensors may include, but are not limited to, accelerometers, gyroscopes, and global positioning system (GPS) receivers. While positional sensors 172 may be an optional component in some versions of the monitor 150, they allow a patient's overall level of activity to be determined, for example, when an accelerometer is used as a pedometer. Additionally, if the orientation of the monitor 150 in space changes radically while the monitor 150 is still in physical contact with skin, that may indicate a fall or another condition in which an alarm should be raised.

In some cases, if one of the positional sensors 172 is a GPS receiver, the monitor 150 may be used to track the location of a patient, which can be particularly valuable with geriatric patients who may be suffering from Alzheimer's disease or other forms of dementia. Even if a GPS receiver is not one of the positional sensors 172 installed in monitor 150, a rough position may be calculated by the monitor 150 itself or by an external device based on data from the cellular network with which the monitor is in communication, or by other means that are known in the art. In that sense, a method of monitoring a patient using a GPS-enabled monitor 172 (or a monitor 150 whose position can be otherwise established) would also include checking the position of the monitor 150 and causing an alarm, either locally at the monitor 150 or at a remote device or station if the monitor 150 has moved beyond a defined area.

While the invention has been described with respect to certain embodiments, the description is intended to be exemplary, rather than limiting. Modifications and changes may be made within the scope of the invention, which is set forth in the following claims.

What is claimed is:

1. A physiological monitor, comprising:
a neck band having the general shape of an elongated horseshoe adapted to be seated around the base of the neck with respective left and right front ends that extend inwardly;
an electrocardiography (EKG) circuit in the neck band, the EKG circuit including at least three leads;
at least one other vital sign sensor within the neck band, such that the physiological monitor acquires both an EKG and at least one other vital sign when the neck band is around the base of the neck;
a processing unit in communication with the EKG circuit and the at least one vital sign sensor;
a wireless transceiver in communication with the processing unit; and
a battery.

2. The physiological monitor of claim 1, wherein the wireless transceiver is adapted to connect the physiological monitor with a cellular communications network.

3. The physiological monitor of claim 1, wherein the at least one other vital sign sensor is selected from the group consisting of a pulse oximeter, a temperature sensor, and a respiration sensor.

4. The physiological monitor of claim 1, wherein the EKG circuit also includes a right leg drive (RLD) electrode.

5. The physiological monitor of claim 1, further comprising one or more touch-sensitive areas on respective inner and outer surfaces of the neck band.

6. The physiological monitor of claim 5, wherein the touch-sensitive areas on the inner surface of the neck band are adapted to be in contact with skin when the neck band is seated around the base of the neck, such that contact between the inner surface of the neck band and skin confirms proper placement of the neck band.

7. The physiological monitor of claim 6, wherein skin contact with the touch-sensitive areas on the inner surface of the neck band turn the physiological monitor on and off.

8. The physiological monitor of claim 7, wherein at least portions of the outer surface of the neck band are adapted to allow the user to control the physiological monitor with one or both of touches or gestures.

9. The physiological monitor of claim 4, further comprising a haptic feedback element connected to the processing unit.

10. The physiological monitor of claim 1, wherein the at least three leads and the at least one other vital sign sensor are provided in a pair of sensor areas provided along an inner perimeter of the neck band opposite one another, in positions to contact sides of a wearer's neck.

11. The physiological monitor of claim 10, wherein the EKG circuit also includes a right leg drive (RLD) electrode and the at least one other vital sign sensor comprises a plurality of sensors including:
a pulse oximeter;
a temperature sensor; and
a respiration sensor.

12. The physiological monitor of claim 11, wherein the wireless transceiver is adapted to connect the physiological monitor with a cellular communications network.

13. The physiological monitor of claim 1, further comprising at least one environmental sensor.

14. The physiological monitor of claim 11, wherein the at least one environmental sensor comprises a gas sensor.

15. The physiological monitor of claim 11, wherein the at least one environmental sensor comprises a UV sensor.

16. The physiological monitor of claim 1, further comprising at least one positional sensor.

17. The physiological monitor of claim 16, wherein the positional sensor comprises an accelerometer, a gyroscope, or a global positioning system (GPS) receiver.

18. The physiological monitor of claim 1, wherein the wireless transceiver comprises a Bluetooth or Wifi transceiver.

19. A method for long-term monitoring of a patient:
reading an electrocardiogram (EKG) signal and at least one other vital sign or signal using a physiological monitor that comprises an encircling neck band adapted to rest around the base of the neck with right and left forward ends that extend inwardly; and
transmitting the EKG signal and the at least one other vital sign to another device using a wireless transceiver integrated into the physiological monitor.

20. The method of claim 19, wherein the transmitting occurs periodically.

21. The method of claim 19, wherein the other device comprises a personal computing device.

22. The method of claim 19, wherein the other device comprises a remote computing device.
